(19) United States
(12) Patent Application Publication
(10) Pub. No.: US 2020/0179598 A1
(43) Pub. Date: Jun. 11, 2020

(54) SIMULTANEOUS USE OF IMAGING AND ENHANCED NEEDLES OR DEVICES TO IMPROVE SAFETY OF IMPLANTABLE PUMP REFILLS AND TROUBLESHOOTING

(71) Applicant: SAOL INTERNATIONAL LIMITED, HAMILTON (BM)

(72) Inventors: David PENAKE, Atlanta, GA (US); Michael SAULINO, Elkins Park, PA (US)

(73) Assignee: SAOL INTERNATIONAL LIMITED, HAMILTON (BM)

(21) Appl. No.: 16/637,114

(22) PCT Filed: Aug. 7, 2018
(86) PCT No.: PCT/US18/45516

§ 371 Date: Feb. 6, 2020

Related U.S. Application Data
(60) Provisional application No. 62/541,904, filed on Aug. 7, 2017.

(51) Int. Cl. A61M 5/168; A61M 5/142; A61M 5/42; A61M 5/32

(52) U.S. Cl. CPC A61M 5/16809; A61M 5/14276; A61M 5/427; A61M 2209/045; A61M 2005/14284; A61M 2205/195; A61M 2205/3375; A61M 5/329

(57) ABSTRACT

Method of safely refilling or accessing a reservoir of an implanted fluid delivery device. The method includes guiding a bent or angled needle into the reservoir while observing an image the implanted fluid delivery device and with the bent or angled needle being oriented in a direction that faces away from an imaging device probe oriented in a substantially vertical orientation and overlying the implanted fluid delivery device.

### CROSS-REFERENCE TO RELATED APPLICATIONS

The instant application is a PCT International Application claiming the benefit of U.S. provisional application No. 62/541,904 filed on Aug. 7, 2017, the disclosure of which is hereby expressly incorporated by reference thereto in its entirety.

### BACKGROUND OF THE INVENTION

Implantable infusion pumps have been in-use and approved in medicine since the 1990s. These pumps allow for a continuous release of medicine to into the body to improve efficacy and reduce side-effects of drugs approved for oral or injectable administration. Implantable infusion pumps solve several important issues when compared with oral or injectable administration. These include the following, for example: 1) Reducing the need to remember to take medication; 2) Particularly for drugs that are used multiple times each day, infusion pumps create a constant stream of medication rather than the typical shark tooth pharmacokinetic profile typical or instantaneous dosing medications; 3) Reducing the overall dose used due to the targeted nature of delivery since medicine is only released to the area where the port or catheter is placed; and 4) May improve the overall efficacy of the therapy, for example, by administering drugs such as baclofen, morphine sulfate, tizanidine, floxuridine, methotrexate, hydromorphone, ziconotide, bupivicaine, clonidine, fentanyl (off-label) and nusinersen, which may be used for targeted drug delivery.

Implantable infusion pumps provide a benefit over external pumps by reducing the likelihood of infection if the intention is a chronic use. Several companies have created, gained approval for, and marketed implantable infusion pump technologies and a few important clinical concerns remain that limit the market potential of this form of targeted chronic therapy.

One such challenge is the risk of a “pocket-fill.” A pocket-fill is what occurs if the needle that enters the implantable pump refill port (subcutaneously through the patient's skin) is either not in the port, falls out of the port, or causes a leak during the refill process. This is a significant clinical issue as pumps are typically filled at a frequency of once every 3 to 6 months. Therefore, a mistake could lead to a half of a year's worth of medication being injected subcutaneously at once. This can lead to severe complications, including death.

Due to this challenge, techniques are used by physicians to reduce the risk of a pocket fill. These include but are not limited to: training, the use of pump templates that allow for a targeted needle placement, palpation, imaging, observation of aspirated fluid and experience that may allow healthcare practitioners to suspect a potential risk.

An attempt to reduce the risk of a pocket fill is described in an Article in Pain Medicine 2011, entitled Ultrasound-Guided Intrathecal Pump Access and Prevention of the Pocket Fill by Michael Gofeld, MD et al., the disclosure of which is herein expressly incorporated by reference in its entirety.

### SUMMARY OF THE INVENTION

The techniques, systems, or methods described in the prior art, however, have deficiencies. Exemplary aspects of the invention are set forth in the claims and pertain to an imaging method or system that address one or more of the deficiencies of the prior art.

One such imaging method that can reduce the risk is the use of ultrasound during the refill procedure. This has significant benefits in assisting with pump-septum identification and attempted confirmation of placement. A key limitation of ultrasound is that technology provides significantly better resolution only when placed directly over imaging site perpendicularly to maximize probe/dermis contact surface area (viewing is best perpendicular to the skin). When outside of this direct line of site, image quality degrades and it becomes more challenging to verify proper positioning with ultrasound.

This challenge is a fundamental problem with the use of implantable infusion pumps because the pump is implanted and sutured into place in a way to make the pump port most easily accessible.

This typically means that needles that are placed directly into the pump port will remain perpendicular to the skin, directly displacing the ability to use ultrasound as a confirmatory imaging technique and reducing the ability to utilize imaging to watch the needle enter the pump septum.

Embodiments of the invention relate to a method of solving this issue as it relates to utilizing simultaneous ultrasound during needle placement and later confirmation of placement and removal when refilling an implantable infusion pump.

The invention proposes the use of a bent, curved, or curvilinear needle that will allow the healthcare practitioner to enter the subcutaneous space away from the perpendicular site directly above, for example, a reservoir and a pump septum (or other seal) that seals the reservoir. This further allows for the continuous use of imaging, such as ultrasound, to verify needle placement, verify that the needle remains in the pump septum (even after being accidentally bumped or jostled from practitioner error or patient movement), and verify that the needle leaves the septum at the end of the refill procedure.

The invention may also help with pump troubleshooting techniques that require catheter port aspiration, which presents a similar challenge.

The invention may be embodied in methods including, but not limited to one of more (and preferably at least two or more) of the following:

The use of any imaging technique (including ultrasound) that provides the greatest image clarity and definition when directly over the targeted image site;
The use of any form of a bent needle, regardless of where the needle is bent;
The use of a curved needle of any curvature;
The use of a curvilinear needle;
The use of a Huber, non-coring needle embodied above;
The use of a needle of any type that can deliver fluid or gas;
The use of a needle of any length;
The use of a needle device combination that allows for non-perpendicular access to a subcutaneous space;
The use of a needle-hose/tube combination that allows for non-perpendicular access to a subcutaneous space;
The use of a needle that can be pre-formed;
The use of a needle that can be bent or reformed during a procedure;
The use of products that enhance imaging quality;
The specific use of ultrasonic gel to enhance the imaging quality of ultrasound;
The use of components that assist in safety of the procedure;
The specific use of sterile components, such as transducer covers to ensure safety of the procedure;
The use of cleaning agents that enhance the safety or improve the image quality of ultrasound;
The use of a disposable imaging method;
The use of imaging that includes a device in combination with a wireless device;
The use of a kit that includes all the necessary pieces to conduct such an imaging technique; and/or
The use of a kit that also includes all the necessary pieces to conduct a pump refill procedure.

An exemplary embodiment of the invention is a method of safely refilling a reservoir of an implanted fluid delivery device, the method comprising guiding a bent or angled needle into the reservoir while observing an image of the implanted fluid delivery device and with the bent or angled needle being oriented in a direction that faces away from an imaging device probe oriented in a substantially vertical orientation and overlying the implanted fluid delivery device. For example, the probe may be tilted in a direction away from a proximal end (e.g., holding portion, conduit, or hose) of the needle.

Another exemplary embodiment of the invention is a method of preventing pocket fill during refilling or accessing a reservoir of an implanted fluid delivery device, the method comprising positioning an imaging device probe in a tilted and substantially vertical orientation and overlying the implanted fluid delivery device in order to obtain an image of the implanted fluid delivery device and locate, for example, the septum (or other seal) and reservoir, and guiding a bent or angled needle into the reservoir by penetrating the septum (e.g., a self-sealing septum or other seal) while observing the image and with the bent or angled needle being oriented in a direction that faces away from the probe. According to the above aspects the approach/advance of the needle (e.g., toward a septum or other seal) may be viewed continuously or progressively as the needle enters a subcutaneous space.

Another exemplary embodiment of the invention is a method of safely accessing a reservoir of an implanted fluid delivery device, the method comprising positioning an imaging device probe in a substantially vertical orientation and overlying the implanted fluid delivery device in order to obtain an image of the implanted fluid delivery device and locate the septum (or other seal) and reservoir, and guiding a bent or angled needle into the reservoir by penetrating the septum (e.g., a self-sealing septum or other seal) while observing the image and with the bent or angled needle being oriented in a direction that faces away from the probe.

In embodiments of the invention, the substantially vertical position may be an angle of between, for example, 0 and 30; 0 and 15; or 0 and 10 degrees relative to an imaginary vertical center axis of the reservoir. For example, when the imaging device probe is in an exactly relative vertical orientation with respect to the reservoir of the implanted fluid delivery device a central axis of the probe may coincide with the imaginary vertical center axis of the reservoir.

In embodiments of embodiment of the invention, the incident angle is between 0 and 5 degrees relative to the imaginary vertical center axis of the reservoir.

In embodiments of the invention, the maximum probe incident angle θᵢ may be between 8 and 12 degrees depending on the depth of the implant relative to the imaginary vertical center axis of the reservoir to provide a quality image of the septum to support reproducible results.

In embodiments of embodiment of the invention, the oriented direction or rotation angle may be an angle of between 0 and 45 degrees relative to an imaginary vertical plane bisecting the probe, with between 0 and 30 degrees being acceptable, and 0 and 15 degrees being preferred

In embodiments of embodiment of the invention, the bent or angled needle is bent or angled at an angle of between 90 and 150 degrees.

In embodiments of embodiment of the invention, the probe is an ultrasound transducer.

In embodiments of embodiment of the invention, the implanted fluid delivery device is an implanted infusion pump.

In embodiments of the invention, there is provided a method of safely refilling a reservoir of an implanted fluid delivery device in a manner that prevents pocket fill, the method comprising positioning an imaging device probe in contact with a skin surface and in a substantially vertical orientation overlying (e.g., partially) the implanted fluid delivery device, obtaining an image of the implanted fluid delivery device and locating the septum (or other seal) and reservoir in the image, and guiding a bent or angled needle into the reservoir while observing the image and with the bent or angled needle being oriented in a direction that faces away from the probe. For example, the probe may be tilted in a direction away from the proximal end (e.g., holding portion, conduit or hose) of the needle.

In embodiments of embodiment of the invention, there is provided a method of preventing pocket fill during refilling or accessing a reservoir (via, e.g., a septum or other seal) of a fluid delivery device implanted in a patient, the method comprising positioning an ultrasonic transducer in a substantially vertical orientation on a patient in a manner which overlies the implanted fluid delivery device in order to obtain an image of the implanted fluid delivery device and locate the reservoir in said image, and during the positioning, guiding a bent or angled needle into the reservoir by penetrating the septum (or other seal) while observing the image and while the bent or angled needle is oriented in a direction that faces away from the probe and within a predetermined angular range. For example, the probe may be tilted in a direction away from a proximal end (e.g., holding portion, conduit or hose) of the needle.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

The figures are intended to show basic features, functioning and aid in understanding and may or may not show features or details which are utilized in commercial embodiments.

FIG. 1A shows a technique of imaging the insertion of a bent or angle needle into an implanted infusion pump in accordance with a non-limiting embodiment of the invention in which the imaging probe is tilted at a predetermined tilt angle range while a bent or angle needle is oriented away from the probe at a predetermined angular range;

FIG. 1B, is a schematic of the physics that guide generalized ultrasonic imaging techniques that may be utilized in imaging an implanted infusion pump.

FIG. 2 shows how the bent or angle needle is guided into a reservoir of the implanted device in a manner which prevents pocket fill.

FIG. 3 shows a top (or plan) view of FIG. 1A and illustrates an orientation or rotation angle of the bent or angle needle at an orientation in which the needle is aligned with an imaginary vertical plane bisecting the probe.

FIG. 4 shows a top view of FIG. 1A and illustrates a rotation angle in one direction designated as a first maximum angle.

FIG. 5 shows a top view of FIG. 1A and illustrates a rotation angle in an opposite direction designated as a second maximum angle.

FIG. 6 illustrates an exemplary method of accessing or refilling a reservoir of an implanted device with a needle in accordance with a non-limiting embodiment of the invention.

FIG. 7 shows exemplary needles that can be inserted into an implanted infusion pump in accordance with the invention.

FIG. 8 illustrates a flow chart detailing how the bent or angle needle may be guided into the reservoir of the implanted device.

FIG. 9 shows an exemplary needle that can be inserted into an implanted infusion pump in accordance with the invention.

FIG. 10 shows a top view of a schematic profile of an implanted infusion pump in accordance with the invention.

FIG. 11 shows a perspective view of implanted infusion pump that may be used in accordance with the invention.

FIG. 12 shows a perspective sectional view of the implanted infusion pump in FIG. 11.

FIG. 13 illustrates a conventional straight needle being inserted substantially perpendicular to the skin surface and reservoir in order to access or refill the reservoir.

### DETAILED DESCRIPTION OF THE INVENTION

The present invention is further described in the detailed description which follows, in reference to exemplary embodiments.

### Non-Limiting Embodiments

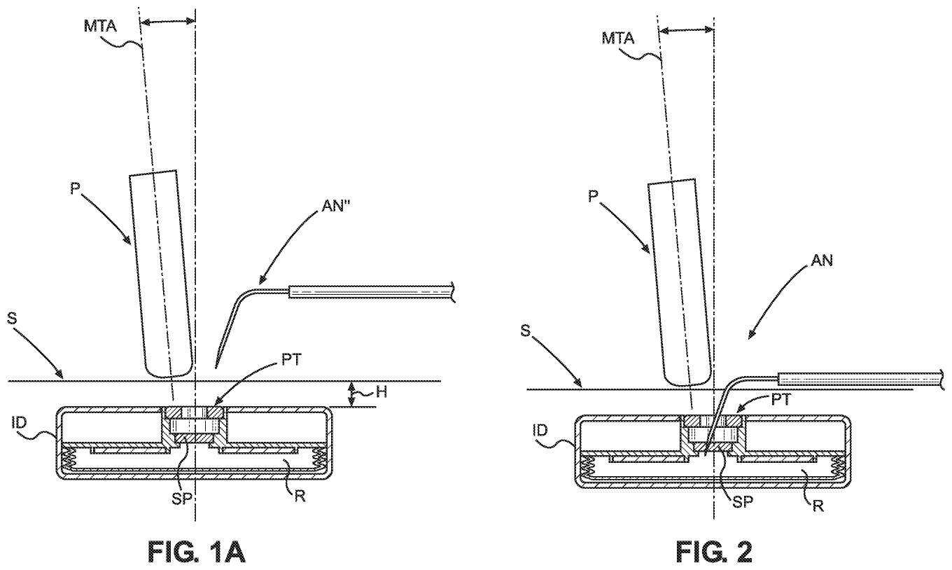

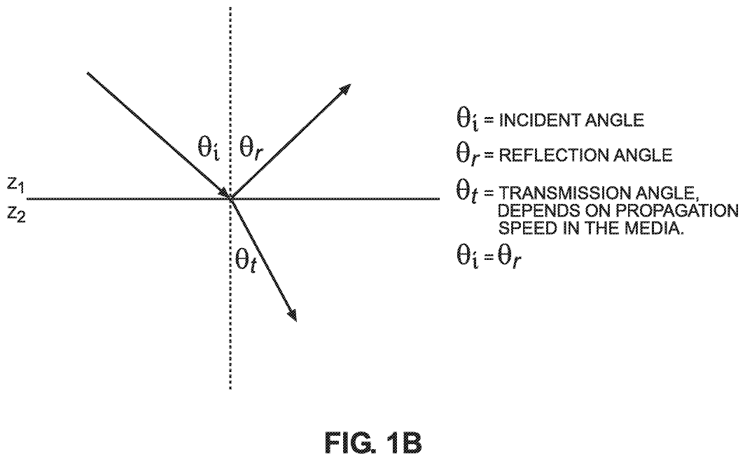

With reference to FIGS. 1-5, there is illustrated an exemplary technique or arrangement for a method of preventing pocket fill during refilling or accessing a reservoir of an implanted fluid delivery device. In the example, the implanted device ID is an infusion pump having a reservoir R which will store and release medicine. The contents of the reservoir R may be sealed by a septum SP (e.g., a self-sealing septum made of material that is capable of resealing itself upon removal of needle insertion, such as a self-sealing polymer material; or other seal). The device ID is typically implanted under the skin S of a patient (animal or human) The device ID may be implanted from, for example, up to several millimeters to several inches beneath the surface of the skin S. As such devices are known, the details of its functioning are not herein described in detail. Although the general location of the device ID is typically well known or can be readily determined, precisely locating, for example, the septum SP (or other seal) and reservoir R of the same can be prone to error—especially when one is seeking to insert a needle within the reservoir R by, for example, penetrating the septum SP.

Further, by inserting the needle AN into the septum SP at an inclination or with a curved or curvilinear profile, the septum SP may be securely penetrated without causing rupture to the septum SP (or other seal). Thus, the methodology of the present disclosure may avoid a straight downward penetrating force that increases a likelihood of rupturing a septum SP or otherwise damaging a pump.

The inventive technique shown in FIGS. 1-5 aims to reduce or eliminate such error by controlling or regulating a number of aspects. The first aspect relates to the positioning of the probe P, which in the exemplary embodiment is an ultrasonic transducer. In order to image the implanted device ID and locate the septum SP (or other seal) and the reservoir R of the same, the probe P should be oriented substantially vertically or slightly tilted at a predetermined maximum angular range MTA. The angle MTA can be measure relative to an imaginary center line (see vertical broken line in FIGS. 1 and 2) passing through the reservoir R and can be between zero degrees and 45 degrees. In embodiments, the angle can be any whole number value within this range as well as any fractional or decimal value such as, e.g., 7.25 or 7 and ¼ degrees. The considerations for controlling this first aspect include positioning the probe P over the device ID and with the aim of obtaining a sufficiently discernable image of the reservoir R (which may be sealed by a septum of other seal). However, in some embodiments, the probe P may not completely overly the reservoir R (and septum SP, e.g., in instances where the reservoir R is sealed by a septum or other seal) as it could prevent the insertion of the needle AN. Instead, it should be laterally offset to one side (e.g., laterally offset from the imaginary center line passing through a center of the reservoir R and septum SP). Non-limiting ranges for the angle MTA include from zero to 45 degrees, with from zero to 30 being acceptable, and from zero to 15 degrees being preferred.

Further FIG. 1B is a schematic illustration of an imaging technique that may be utilized in imaging an implanted device. The imaging technique of FIG. 1B may be utilized to facilitate insertion of a needle AN into a reservoir (e.g., by penetrating a septum or other seal) in accordance with aspects of the present disclosure. For example, according to other aspects of the disclosure a preferred range for θᵢ may be from 8 to 12 degrees in order to obtain a clear image of the reservoir R (and septum or other seal) and view the approach/advance of and insertion of the needle AN into the reservoir R. It is noted that in FIG. 1B, θᵣ denotes a reflection angle, which, theoretically, should be approximately equal to θᵢ; θₜ denotes a transmission angle that is influenced by, or depends upon, a propagation speed in the medium.

The second aspect relates to the type of needle AN used, which in the exemplary embodiment is a bent or angle needle AN. In order that the puncturing end of the needle AN decent or insert into the reservoir R correctly, it may be desired that the needle is inserted transverse to the reservoir R. Thus, the needle AN should be bent or angled so that the inserted portion of the same enters the reservoir R properly, for example, by penetrating a septum SP (or other seal) at an inclination or with a curved or curvilinear profile. Of course, if the probe P is improperly tilted or positioned, there can be a risk that the needle AN will not properly be inserted. As such, controlling both the first and second aspects should be an aim. An opposite end of the needle AN is typically connected to a supply of medicine via conduit or hose. Non-limiting examples of the needle AN include a Huber type needle. Orienting the angle needle AN away from the probe P allows one to grip the needle AN more securely and with less risk of the probe P interfering with the same. For example, the probe may be tilted in a direction away from a proximal end (e.g., holding portion, conduit or hose) of the needle.

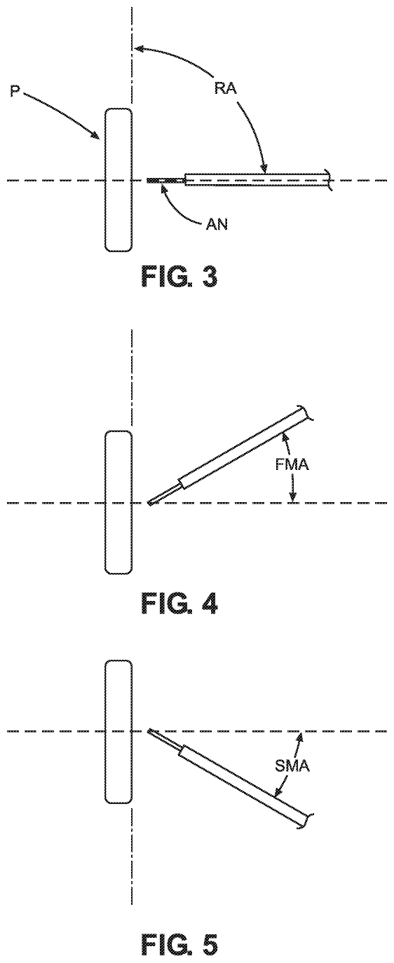

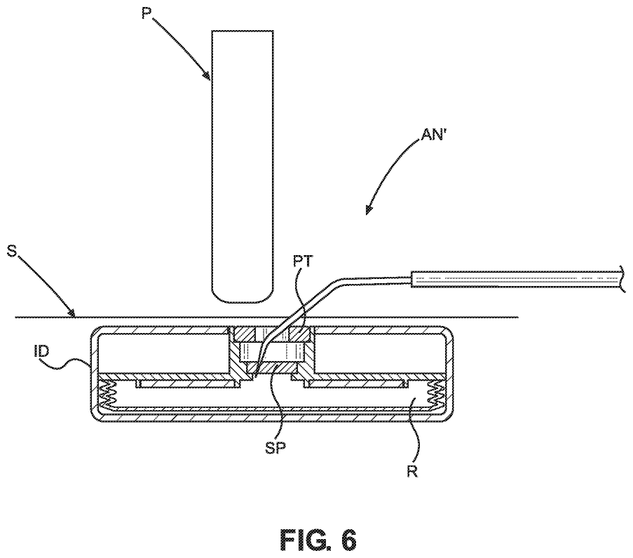

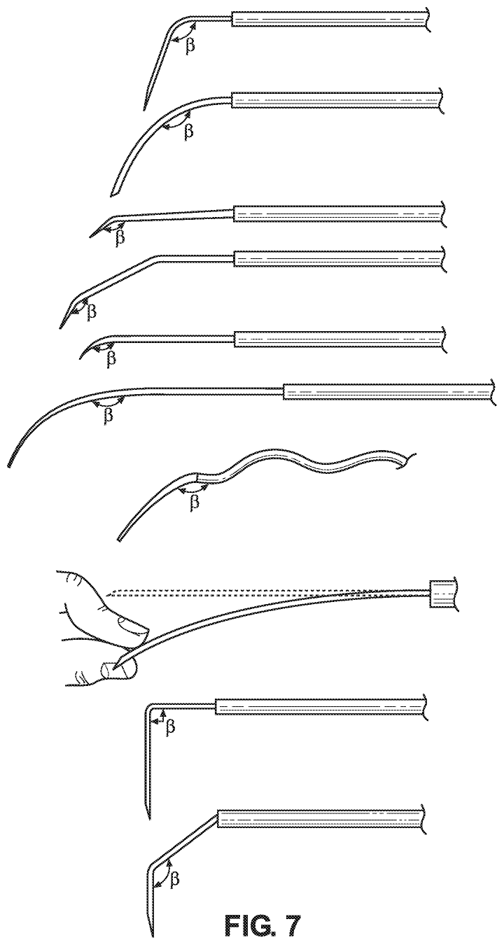

The third aspect relates to the orientation or rotation angle RA of needle AN, which in the exemplary embodiment shown in FIGS. 3-5 can be such that the needle is aligned with an imaginary vertical plane bisecting the probe as shown in FIG. 3 (e.g., with RA being an angle formed between an imaginary vertical plane bisecting the probe and a reference plane extending perpendicular to the imaginary vertical plane bisecting the probe, the reference plane coinciding with a side of the probe facing the needle AN) to either of two maximum rotation angles from the imaginary vertical plane bisecting the probe. In FIG. 3 RA is illustrated at 90 degrees. The two maximum rotation angles include a first maximum angle FMA in one direction as shown in FIG. 4 and a second maximum angle SMA in an opposite direction as shown in FIG. 5. The illustrated needle orientation allows the non-puncturing end of the needle AN to face in a direction that is opposite from the tilt direction of the probe B. Non-limiting ranges for the angle FMA and/or the angle SMA include from zero to 45 degrees, with from zero to 30 being acceptable, and from zero to 15 degrees being preferred. In other words, the preferred orientation of the needle allows for the needle to be firmly manipulated at a distance from the site where the needle enters the subcutaneous space, thereby allowing a surgeon to perform the procedure without interference from the probe P.

With reference again to FIGS. 1A and 2, the technique will now be described. When the probe P is positioned and oriented as shown in FIG. 1A, one can obtain an image of the implanted device ID and locate the reservoir R (and, for example, a septum SP or other seal that seals the reservoir R). At this point, one can position the angled needle AN near the probe P and begin to insert the same into the skin S. The imaging data obtained by the probe P can be monitored on a display (not shown) to obtain a visual indication that the needle AN has been correctly inserted into the reservoir R as shown in FIG. 2 (e.g., by penetrating a septum, or other seal, at an inclination or with a curved or curvilinear profile). Thus, the technique involves the positioning of an imaging device probe P in a tilted and/or substantially vertical orientation and overlying the implanted fluid delivery device ID in order to obtain an image of the implanted fluid delivery device ID and locate the reservoir R and septum SP (or other seal). Next, a bent or angled needle AN is guided into the reservoir R (e.g., by penetrating a septum SP or other seal) while observing the image and with the bent or angled needle being oriented in a direction that faces away from the probe.

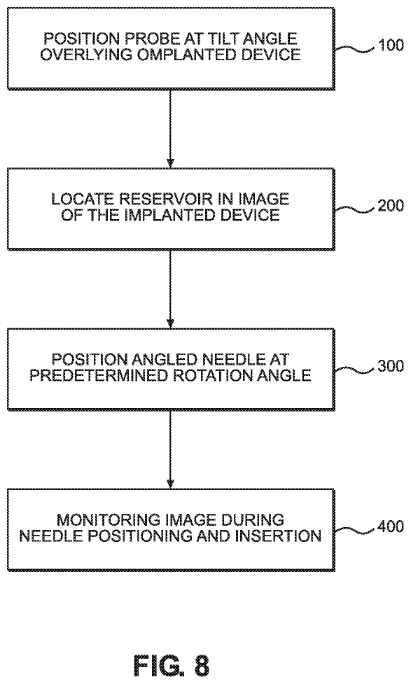

With reference to FIG. 8, as well as FIGS. 1 and 2, an exemplary method of implementing the technique will now be described. In a first stage 100 of the method, the probe P is positioned on the skin so that the probe P assumes a tilted position overlying the implanted device ID with attention being paid to controlling the tile angle within a pre-specified range and so as not to block the insertion of the needle AN into the reservoir R. The correct position will be seen in the image obtained by the probe P when the reservoir R is located in a second stage 200 of the method. In a third stage 300 of the method, an angled needle AN is positioned on the skin so that the needle AN overlies the reservoir R with attention being paid to controlling the rotation orientation angle within a pre-specified range and so as to allow safe insertion of the needle AN into the reservoir R. The correct needle insertion positioning will be seen in the image obtained by the probe P when the reservoir R receives therein the needle AN in stage 400.

With reference to FIG. 7, one can see that the angle needle AN can have different configurations ranging from one having bends, inclinations, curves, as well as combinations of bends inclinations and curves. The needle may include opposite ends oriented in perpendicular directions to one with ends oriented at an angle (β) that is between 90 and 180 degrees and encompassing any angle in between.

With further reference to FIG. 7, the particular needle may be selected based upon several considerations. For example, in selecting a needle AN one may consider a variety of parameters including but not limited to: the depth H of the implanted device ID (i.e., beneath the skin surface); the positioning of the reservoir R; the diameter, width or other dimension of the septum SP (or other seal); a vertical distance between, a refill port PT and the septum SP. In other words, a needle having a suitable geometry for insertion (e.g., at an inclination or curvilinear manner) into the reservoir R based on the aforementioned parameters may be selected.

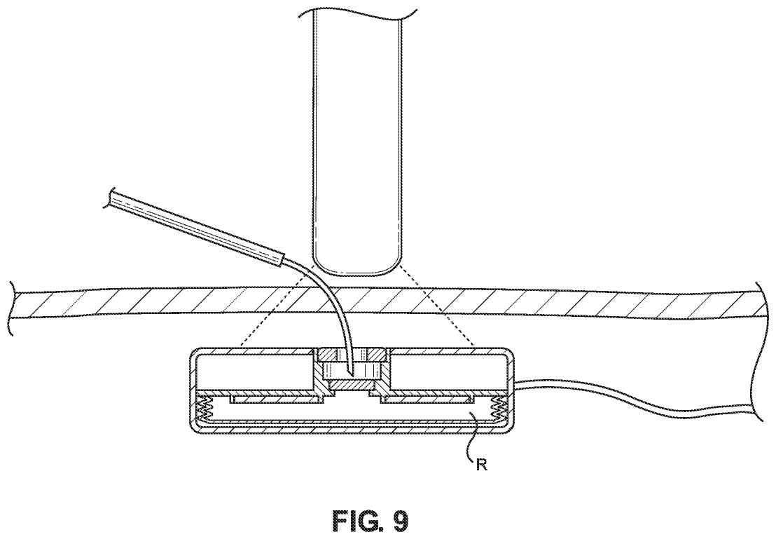

With reference to FIG. 9, for example, a curved needle AN may be selected for insertion into the reservoir R with a gently curved arc. In selecting such a needle AN the above-mentioned parameters may be taken into consideration. Overall, it should be appreciated that a set of needles, for example, as illustrated in FIG. 7 provide for a variety of different selections that may be suitable for refilling an implanted device ID, i.e., given, for example, the placement and geometry of the implanted device.

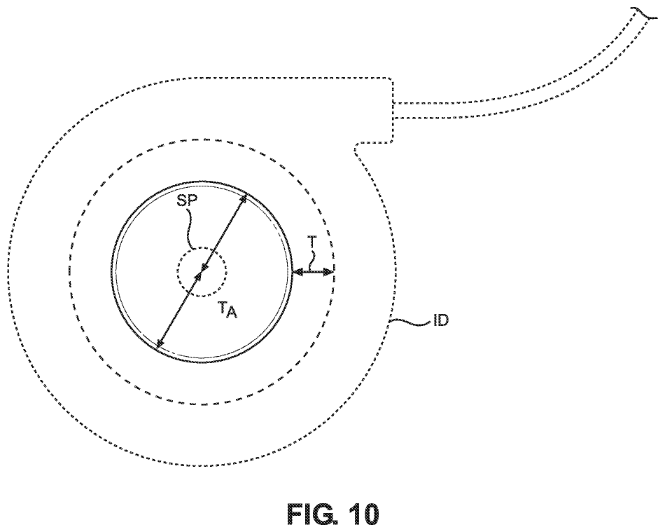

With reference to FIG. 10, a schematic top (or plan) view of the implanted device ID is illustrated. In order to ensure that a needle AN is properly inserted into a reservoir R, the needle may be inserted into the skin surface S at, e.g., an annular region T, which is intermediate an outer profile of the implanted device ID and an inner circular region T_{A}, e.g., encompassing a septum SP. If the needle AN is inserted into the skin (i.e., enters a subcutaneous area) at the inner circular region T_{A}, it may be difficult to view the approach/advance of the needle AN as it is inserted into the reservoir R (via, for example, a septum SP or other seal).

It is noted that the particular regions T (the target area) and T_{A} (which is an area to avoid) are determined based upon, for example, the above discussed parameters including but not limited to: the depth of the implanted device ID (i.e., beneath the skin surface); the positioning of the reservoir R; the diameter, width or other dimension of the septum SP; a vertical distance between, a fill port and the septum SP. In other words, a needle having a suitable geometry for insertion (e.g., at an inclination or curvilinear manner) into the reservoir R based on the aforementioned parameters may be selected.

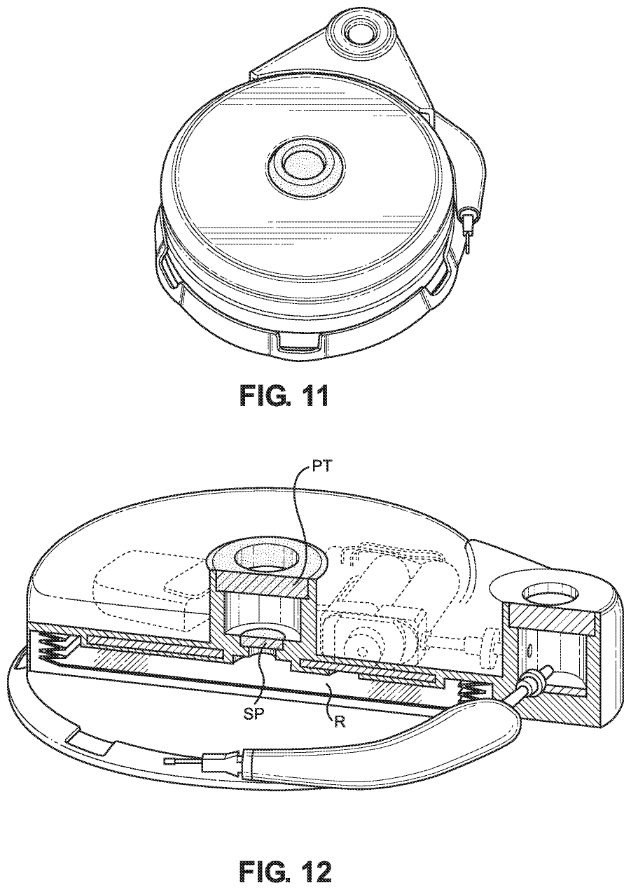

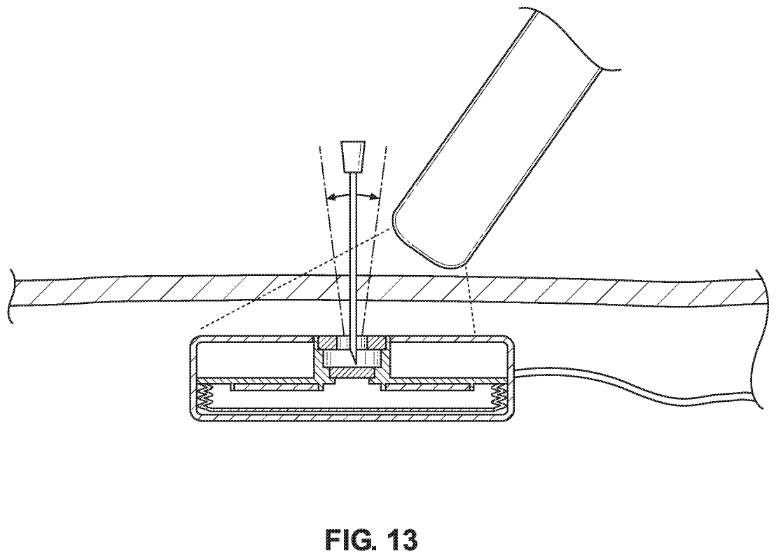

With respect to FIGS. 11 and 12, an exterior/perspective and cross-sectional view of an Implanted Device that may be utilized in accordance with aspects of the present disclosure is illustrated. In FIG. 12, the septum SP is illustrated recessed downwardly from the fill port and sealing the reservoir. However, it should be appreciated that various types of implanted devices that allow for insertion into the reservoir R at an inclination or in a curved or curvilinear manner, or transverse in any other manner, may be utilized. Such an implanted device may have various arrangements of reservoirs R and septum SP (and other seals), as well as other geometries.

There are numerous types of implanted devices that may be used to practice the invention including the SYNCHROMED II®, SYNCHROMED®, FLOWONIX®, PROMETRA® pump, and any other implanted pump suitable for dispensing drugs. While embodiments are not limited to the administration of any particular drug, those that are most commonly used in implanted pumps include, but are not limited to: GABAB receptor agonists, opioids, local anesthetics, adrenergic agonists, N-methyl-D-aspartate receptor agonists, and specifically, for example, nusinersen, tizanidine, floxuridine, methotrexate, hydromorphone, morphine, baclofen, ziconotide, bupivacaine, clonidine, fentanyl, and lidocaine.

### Example A

An exemplary method of the invention includes safely refilling a reservoir R of an implanted fluid delivery device ID, by guiding a bent or angled needle AN into the reservoir R (e.g., by penetrating a septum of other seal) while observing an image of the implanted fluid delivery device ID and with the bent or angled needle AN being oriented in a direction that faces away from an imaging device probe P oriented in a substantially vertical orientation and overlying the implanted fluid delivery device.

### Example B

An exemplary method of the invention includes preventing pocket fill during refilling or accessing a reservoir R of an implanted fluid delivery device ID, by positioning an imaging device probe P in a tilted and substantially vertical orientation and overlying the implanted fluid delivery device ID in order to obtain an image of the implanted fluid delivery device ID and locate the reservoir R and guiding a bent or angled needle AN into the reservoir R (e.g., by penetrating a septum SP or other seal) while observing the image and with the bent or angled needle AN being oriented in a direction that faces away from the probe P.

### Example C

An exemplary method of the invention includes safely accessing a reservoir R of an implanted fluid delivery device ID, by positioning an imaging device probe P in a substantially vertical orientation and overlying the implanted fluid delivery device ID in order to obtain an image of the implanted fluid delivery device ID and locate the reservoir R (e.g., by penetrating a septum SP or other seal). The method also includes guiding a bent or angled needle AN into the reservoir R while observing the image and with the bent or angled needle AN being oriented in a direction that faces away from the probe P.

### Example D

An exemplary method of the invention includes safely refilling a reservoir R of an implanted fluid delivery device ID in a manner that prevents pocket fill, by positioning an imaging device probe P in contact with a skin surface S and in a substantially vertical orientation overlying the implanted fluid delivery device ID, obtaining an image of the implanted fluid delivery device ID and locating the reservoir R in said image, and guiding a bent or angled needle AN into the reservoir R (e.g., by penetrating a septum SP or other seal) while observing the image and with the bent or angled needle AN being oriented in a direction that faces away from the probe P.

### Example E

An exemplary method of the invention includes preventing pocket fill during refilling or accessing a reservoir R of a fluid delivery device ID implanted in a patient, by positioning an ultrasonic transducer P in a substantially vertical orientation on a patient in a manner which overlies the implanted fluid delivery device ID in order to obtain an image of the implanted fluid delivery device ID and locate the reservoir R in said image, and during the positioning, guiding a bent or angled needle AN into the reservoir R (e.g., by penetrating a septum SP or other seal) while observing the image and while the bent or angled needle AN is oriented in a direction that faces away from the probe P and within a predetermined angular range.

The devices and methods described above can also utilize one or more features disclosed in the prior art documents expressly incorporated by reference herein.

At least because the invention is disclosed herein in a manner that enables one to make and use it, by virtue of the disclosure of particular exemplary embodiments of the invention, the invention can be practiced in the absence of any additional stage, element or additional structure that is not specifically disclosed herein.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A method of safely refilling a reservoir of an implanted fluid delivery device, the method comprising:
guiding a bent or angled needle into the reservoir while observing an image the implanted fluid delivery device and with the bent or angled needle being oriented in a direction that faces away from an imaging device probe oriented in a substantially vertical orientation and overlying the implanted fluid delivery device.

2. A method of preventing pocket fill during refilling or accessing a reservoir of an implanted fluid delivery device, the method comprising:
positioning an imaging device probe in a tilted and substantially vertical orientation and overlying the implanted fluid delivery device in order to obtain an image of the implanted fluid delivery device and locate the reservoir; and
guiding a bent or angled needle into the reservoir while observing the image and with the bent or angled needle being oriented in a direction that faces away from the probe.

3. A method of safely accessing a reservoir of an implanted fluid delivery device, the method comprising:
positioning an imaging device probe in a substantially vertical orientation and overlying the implanted fluid delivery device in order to obtain an image of the implanted fluid delivery device and locate the reservoir; and
guiding a bent or angled needle into the reservoir while observing the image and with the bent or angled needle being oriented in a direction that faces away from the probe.

4. The method of claim 3, wherein the substantially vertical position is an angle of between 0 and 30 degrees relative to an imaginary vertical center axis of the reservoir.

5. The method of claim 4, wherein the angle is between 0 and 10 degrees relative to the imaginary vertical center axis of the reservoir.

6. The method of claim 3, wherein the oriented direction is an angle of between 0 and 45 degrees relative to an imaginary vertical plane bisecting the probe.

7. The method of claim 3, wherein the bent or angled needle is bent or angled at an angle of between 90 and 150 degrees.

8. The method of claim 3, wherein the probe is an ultrasound transducer or integrated ultrasound device.

9. The method of claim 3, wherein the implanted fluid delivery device is an implanted infusion pump.

10. A method of safely refilling a reservoir of an implanted fluid delivery device in a manner that prevents pocket fill, the method comprising:
positioning an imaging device probe in contact with a skin surface and in a substantially vertical orientation overlying the implanted fluid delivery device;
obtaining an image of the implanted fluid delivery device and locating the reservoir in said image; and
guiding a bent or angled needle into the reservoir while observing the image and with the bent or angled needle being oriented in a direction that faces away from the probe.

11. A method of preventing pocket fill during refilling or accessing a reservoir of a fluid delivery device implanted in a patient, the method comprising:
positioning an ultrasonic transducer in a substantially vertical orientation on a patient in a manner which overlies the implanted fluid delivery device in order to obtain an image of the implanted fluid delivery device and locate the reservoir in said image; and
during the positioning, guiding a bent or angled needle into the reservoir while observing the image and while the bent or angled needle is oriented in a direction that faces away from the probe and within a predetermined angular range.

12. The method of claim 3, further comprising a seal that seals the contents of the reservoir, and the needle penetrating the seal at an inclination or with a curvilinear profile.

13. The method of claim 12, wherein the seal is provided as a self-sealing septum.

14. The method of claim 12, further comprising positioning the imaging device probe to partially overlay the septum in the substantially vertical orientation.
